(19) United States
(12) Patent Application Publication
(10) Pub. No.: US 2022/0015858 A1
(43) Pub. Date: Jan. 20, 2022

(54) SPIKE FOR BONE AXIS DIGITIZER DEVICE

(71) Applicant: ORTHOSOFT ULC, Montreal (CA)

(72) Inventors: Yann DUBOIS, Montreal (CA); Joseph MADIER-VIGNEUX, Montreal (CA)

(21) Appl. No.: 17/378,020

(22) Filed: Jul. 16, 2021

Related U.S. Application Data
(60) Provisional application No. 63/053,031, filed on Jul. 17, 2020.

(51) Int. Cl. A61B 90/00

(52) U.S. Cl. CPC A61B 90/39; A61B 2562/028; A61B 2090/3991; A61B 2090/3916; A61B 2562/0219

(57) ABSTRACT

A spike for a bone axis digitizer device may include a leading member having a pointy end configured for penetrating a bone or cartilage, the leading member defining a penetration axis. An anti-rotation feature may project laterally from a surface of the leading member. The spike has a first penetration segment and a second penetration segment, the first penetration segment including the pointy end and configured for leading a penetration of the spike in the bone or cartilage, and the second penetration segment having the at least one anti-rotation feature. A bone axis digitizer device with the spike may also be provided.

### CROSS-REFERENCE TO RELATED APPLICATION

The present application claims the priority of U.S. Patent Application No. 63/053,031, filed on Jul. 17, 2020 and incorporated herein by reference.

### TECHNICAL FIELD

The present application relates generally to computer-assisted surgery (CAS) systems and, more particularly, to hardware used to align tools with anatomical axes, such as a tibial mechanical axis using such a CAS system.

### BACKGROUND OF THE ART

In computer-assisted surgery (CAS) systems which employ inertial-based or micro-electro-mechanical sensor (MEMS), trackable members continue to be developed. One of the principal steps in navigating a bone with inertial sensors is to determine a coordinate system of the bone relative to the sensors, so as to be able to track the orientation of the bone.

Some bone axis digitizer devices have been used as a structural component to attach to elongated bones and serve as a tracker for the tracking of the orientation of the bone. Such bone axis digitizer devices typically supports MEMS that keep track of an orientation of an axis of the bone. In order to prevent movement of bone axis digitizer devices, multipoint attachments are typically provided as part of bone axis digitizer devices. The multipoint attachments may be bulky, and their installation on the bone may result in slight displacement of the devices relative to the bone. There remains a need for an improved attachment configuration for bone axis digitizer.

### SUMMARY

In one aspect, there is provided a spike for a bone axis digitizer device comprising: a leading member having a pointy end configured for penetrating a bone or cartilage, the leading member defining a penetration axis; and at least one anti-rotation feature projecting laterally from a surface of the leading member; wherein the spike has a first penetration segment and a second penetration segment, the first penetration segment including the pointy end and configured for leading a penetration of the spike in the bone or cartilage, and the second penetration segment having the at least one anti-rotation feature.

In another aspect, there is provided a bone axis digitizer device comprising: a main arm configured to extend along a bone; a clamp at an end portion of the main arm, the clamp configured to clamp to an anatomical portion; an attachment member at another end portion of the main arm; and a spike projecting from the attachment member, the spike including a leading member having a pointy end configured for penetrating a bone or cartilage, the leading member defining a penetration axis, and at least one anti-rotation feature projecting laterally from a surface of the leading member, wherein the spike has a first penetration segment and a second penetration segment, the first penetration segment including the pointy end and configured for leading a penetration of the spike in the bone or cartilage, and the second penetration segment having the at least one anti-rotation feature; wherein the bone axis digitizer device is configured to receive an inertial sensor unit thereon.

In yet another aspect, there is provided a method for installing a bone axis digitizer device on a bone comprising: penetrating a bone and/or cartilage with a pointy end of a spike such that a first penetration segment of the spike penetrates the bone and/or cartilage; adjusting an orientation of the bone axis digitizer device by rotation of the spike relative to the bone; and further penetrating the bone and/or cartilage with a second penetration segment of the spike, the second penetration segment having an anti-rotation feature blocking rotation of the spike relative to the bone.

### DESCRIPTION OF THE DRAWINGS

Reference is now made to the accompanying figures in which:

FIG. 1 is a perspective view of a bone axis digitizer device with a spike in accordance with the present disclosure;

FIG. 2 is an isometric view of the spike on an attachment member of the bone axis digitizer device of FIG. 1;

FIG. 3A is a perspective view showing a first step of attachment of the spike to the bone; and

FIG. 3B is a perspective view of a second step of attachment of the spike to the bone.

### DETAILED DESCRIPTION

The present surgical tool and method will be generally described herein with respect to use of the device in conjunction with an inertial-based computer-assisted surgery (CAS) system employing trackable members having inertial-based sensors, such as the MEMS-based system and method for tracking a reference frame disclosed in U.S. Pat. No. 9,901,405, and the MEMS-based system and method for planning/guiding alterations to a bone disclosed in U.S. Pat. No. 8,265,790, the entire contents of both of which are incorporated herein by reference. The term “MEMS” is used herein to refer to micro-electro-mechanical sensors, for example, but not limited to, accelerometers, gyroscopes and other inertial sensors. However, it is to be understood that the tool and method described herein may also be used with other CAS systems, with other tracking modalities, such as optical tracking.

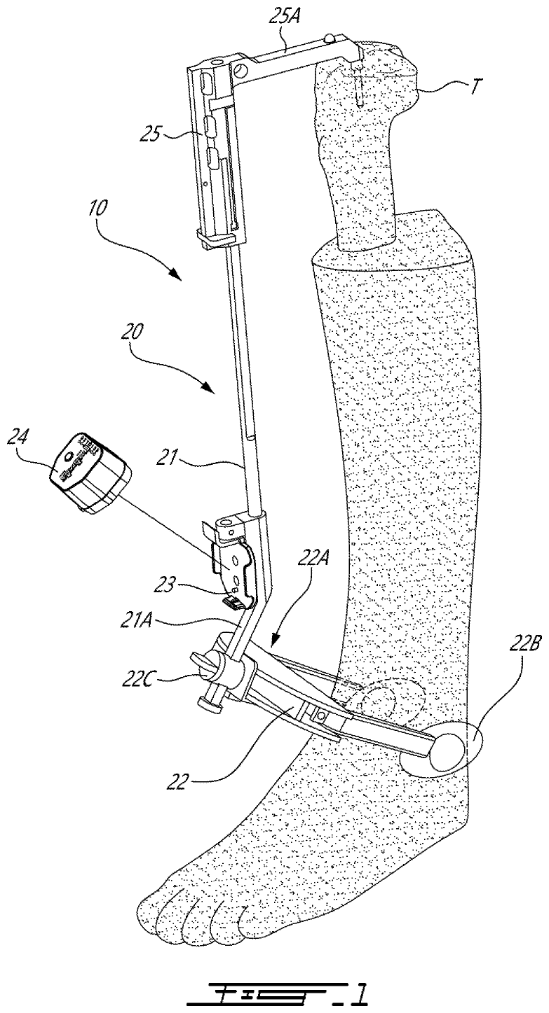

Referring to FIG. 1, a bone axis digitizer device in accordance with the present disclosure is generally shown at 10. The bone axis digitizer device 10 in this embodiment is an exemplary tibial digitizer, which may, in a particular embodiment, be provided for use with an inertial-based CAS system in order to digitally acquire the mechanical axis of the tibia, or other tibial axis, for subsequent tracking relative to the mechanical axis. For instance, the bone axis digitizer device may have a configuration similar to that of U.S. Patent Application Publication No. 2012/0053594, incorporated herein by reference. Thus, as will be described, the tibial digitizer 10 includes trackable members thereon which, in at least the presently described embodiment, include inertial sensors for communication with the inertial-based CAS system. These inertial sensors are referred to as MEMS sensors or MEMS trackable members in the embodiment described below, however it is to be understood that the term “MEMS” or “MEMS sensor” as used herein may include any combination of inertial-based tracking circuitry, for example including MEMS, gyroscopes, accelerometers, compasses, electronic tilt sensors, etc., all of which are able to detect orientation changes. However, although particularly developed for use with inertial based sensors and an inertial-based CAS system, it is also to be understood that the present tibial digitizer may similarly be used with other CAS systems, and thus may include trackable members thereon which are not exclusively inertia-based. As will be described in further detail below, the bone axis digitizer device 10 is used to digitally acquire the mechanical axis of the tibia, in a manner which is quick, accurate and easily repeatable. The bone axis digitizer device 10 may be used with other bones as well, such as the femur, the humerus, among other examples.

The mechanical axis of the tibia T may in fact be defined by two reference points located from known landmarks on the bone. One of these two reference points may be the midpoint between the most medial point on the medial malleolus and the most lateral point of the lateral malleolus (on the fibula) which make up the ankle. Another of these two reference points may be the mechanical axis entry point on the tibial plateau. The generally accepted mechanical axis entry point on the tibial plateau may be used. However, in one particular embodiment, the mechanical axis entry point on the tibial plateau may be defined as being at the intersection of two axes on the tibial plateau, the first axis being centered medial-laterally and the second axis being located one-third anterior and two-thirds posterior, as a possibility among others. Thus, the mechanical axis of the tibia T is defined between the two reference points, which can be located and acquired by the CAS system for the tibia T using the identified anatomical landmarks which are located by the bone axis digitizer device 10. Other anatomical landmarks may be used.

The bone axis digitizer device 10 has a guide frame 20 having a spike 30 in accordance with the present disclosure. The guide frame 20 is used to form a structural reference secured to the bone or in a fixed relation with the bone, and may be used for tracking of the bone in a reference coordinate system, a.k.a., frame of reference. The guide frame 20 may for instance be attached to a bone of a patient in a given orientation, such as being generally parallel to the anatomical axis of the bone. For example, in FIG. 1, the bone axis digitizer device 10 is attached to a tibia T, and may be used to track a mechanical axis of the tibia T. One or more surgical implement, such as a cutting guide, may be attached and supported by the guide frame 20. The cutting guide is used to guide alteration tools, such as a flat saw blade, in the manner described relative to the illustrated embodiment, for resecting the tibia and creating a tibial plateau plane. Other surgical implements or guides could be used, such as a drill guide for a drill among possible tools. Other tools may include a reamer, etc.

Referring to FIG. 1, the guide frame 20 is shown as having a main arm 21. The main arm 21 extends generally along the tibia T when installed onto the leg of the patient, and may be generally parallel to the tibia T. The main arm 21 may be an elongated member, such as a shaft, a rod, etc. There may also be more than one arm. In the tibial embodiment, the main arm 21 may have a lower bend 21A, as a possibility, to follow the anatomy of the lower leg. The lower bend 21A may be a straight segment, with a remainder of the main arm 21 being a straight segment as a possibility as well. An angle between the lower bend 21A, if present, and a remainder of the main arm 21 may be between 5 degrees and 40 degrees, as an example. The main arm 21 may be regarded as a main structural component of the bone axis digitizer device 10 as it supports and interconnects various components of the bone axis digitizer device 10, as described below.

A clamp 22 may be located at a bottom end of the main arm 21. The clamp 22 may be provided to non-invasively attach and fix the guide frame 20 to a user's ankle, in the exemplary embodiment of a tibial digitizer. In another embodiment, the clamp 22 could be used to attach the guide frame 20 to a lower part of the tibia. Other configurations are contemplated. In an embodiment, the clamp 22 has an inverted U frame 22A at the end of which are positioned malleolus cups 22B. The U frame 22A may allow the pivoting or translation motion of the cups 22B for them to be posed onto the malleoli. In an embodiment, the cups 22B are biased toward one another so as to naturally exert pressure and clamp onto the malleoli. Other configurations are considered as well, such as jaws, flat abutments, etc. If the cups 22B are biased, the biasing force should be sufficient to allow a suitable clamping force while not preventing the cups 22B from being manually separated from one another. In an embodiment, the clamp 22 is relatively symmetric to allow the self-centering of the clamp 22 on the portion of the anatomy it will grasp. In another embodiment, the U frame 22A has a joint (e.g., endless crew and racks) that is manually rotated to cause a translation of the cups 22B toward or away from one another, while preserving an equal distance between the cups 22B and an imaginary center between them. The clamp 22 may be mounted to the main arm 21 by a lockable translational joint 22C, for example to be adjusted in position along the main arm 21. As shown, the translational joint 22C may be on the bend segment 21A, and may feature a locking screw. An indexing mechanism is contemplated as well. Therefore, when positioning the guide frame 20 on the limb, the position of the lower part of the guide frame 20 can readily be adjusted by manipulations of the clamp 22 or equivalent, for the clamp 22 to clamp onto the ankle (or other anatomical part) in a self-centering manner.

Other bottom end configurations may be present on the guide frame 20. For example, as an alternative to the U frame 22, it is considered to provide a strap, an elastic, and/or a V-shaped structure or the like, located at the bottom end of the main arm 21. Such configurations are non-invasive as they attach to the surface of the skin, but invasive attachments are considered as well. Moreover, even though FIG. 1 shows a tibia, the bone axis digitizer device 10 may be used with other bones, and the clamp 22 or like attachment implement may be configured as a function of the bone with which the bone axis digitizer device 10 will be used.

A support 23 may be provided on the main arm 21 or on any other portion of the guide frame 20, the support 23 being configured to receive an inertial sensor unit 24 or like MEMS thereon, as one of the possible types of tracking technologies that may be used with the guide frame 20. In an embodiment, the inertial sensor unit 24 is in the form of a pod that is releasably connectable to the support 23. The inertial sensor unit 24 may include a processor and a non-transitory computer-readable memory communicatively coupled to the processor and comprising computer-readable program instructions executable by the processor. Moreover, the inertial sensor unit 24 may be self-contained, in that it is pre-calibrated for operation, has its own powering or may be connected to a power source, and has an interface, such as in the form of a display thereon (e.g., LED indicators). Hence, the bone axis digitizer device 10 may be qualified as being a computer-assisted solution by the presence of the inertial sensor unit(s) 24 alone. It is also considered to have a computerized ecosystem including the inertial sensor unit(s) 24, a monitor, another processing unit, a tablet or like portable hand-held device, etc.

The inertial sensor unit 24 may also be directly integrated onto the guide frame 20, though the releasable configuration may be well suited for preprogramming, sterilization, etc. As the main arm 21 may preferably be oriented in a generally parallel manner to the anatomical axis of the humerus, the positioning of the support 23 on the main arm 21 may facilitate the calibrating of the inertial sensor unit 24. In an embodiment, the interconnection between the support 23 and the inertial sensor unit 24 is such that it is calibrated into the inertial sensor unit. Stated differently, once the inertial sensor unit 24 is in the support 23, the inertial sensor unit 24 may have been pre-calibrated in such a way that a coordinate system maintained and tracked by the inertial sensor unit 24 thereof is aligned with a length of the main arm 21. Accordingly, if the main arm 21 is generally parallel to the tibial mechanical axis, the inertial sensor unit 24 may automatically track the mechanical axis in its XYZ coordinate system. Therefore, in an embodiment, once the inertial sensor unit 24 is turned on, with the guide frame 20 attached to the leg, the inertial sensor unit 24 may continuously track an orientation of the upper arm, in phi, theta, rho (i.e., three rotational degrees of freedom—DOF).

Still referring to FIG. 1, an attachment member 25 may be connected to a top end of the main arm 21. A translational joint may be formed between the main arm 21 and the attachment member 25 so as to expand or contract the guide frame 20, to adapt the guide frame 20 to the user's bone length. A direction of the translational joint may be parallel to a length of the main arm 21. In an embodiment, the translational expansion may be possible by a telescopic joint. In an embodiment, the telescopic joint defines a plurality of indexed positions with appropriate snap-fit indexing features (e.g., spring loaded ball and groove). Other joint configurations may be used, such as endless screw engagement, set screw locking, and/or biasing force to block the movement of the attachment member 25 relative to the main arm 21. A push button or detent may be present to release the lock of the attachment member 25 and allow expansion or contraction of the guide frame 20, by friction for example. In an embodiment, the attachment member 25 forms a female member receiving the main arm 21, the latter acting as a male member 21. The reverse arrangement is possible, or other configuration including a rail and guide, for example.

In an embodiment, a side arm 25A of the attachment member 25 is perpendicular or transverse to the main arm 21, or projects laterally relative to the main arm 21. The side arm 25A may also have a telescopic joint. It is also contemplated to have the side arm 25 be of fixed length as well, as shown. The side arm 25A may therefore be placed in a hovering arrangement over the tibial plateau TP. The side arm 25A may be of adjustable length, with a telescopic joint present therein, for example.

Referring now to FIG. 2, the spike 30 is shown in greater detail. The spike 30 is at a free end of the side arm 25A of the attachment member 25, and is configured to be planted into the bone and/or cartilage in the tibial plateau, or other bones in other applications, along a central axis X thereof, also known or also coincident with the penetration axis, the central axis X being in an embodiment parallel to the longitudinal axis of the main arm 21. In an embodiment, the spike 30 is releasably fixed to the free end of the attachment member 25 such that another type of spike with other dimensions may be used with the bone axis digitizer device 10 based on the application. Hence, the bone axis digitizer device 10 may be universal. In another embodiment, the spike 30 is an integral member of the attachment member 25. The spike 30 may be made of a stiff and hard material such as a metal, or some types of polymers, and/or combinations thereof. The spike 30 may have a monoblock construction.

The spike 30 is thus configured to penetrate the bone and provides an anti-rotation feature so as to preclude or limit rotation of the guide frame 20 once attached to the bone at the attachment member 25. According to an embodiment, the spike 30 has a leading member 31, also referred to as a central member as it is centered in the spike 30. The leading member 31 may be constituted of one or more segments. In an embodiment, one segment is the base 32. The base 32 is shown as having a cylindrical body. Another segment is defined by pointy end 33 connected to the cylindrical base 32. In an embodiment, the base 32 and the pointy end 33 are an integral component. The pointy end 33 may have different shapes, but is shown to have a conical geometry. The pointy end 33 flairs from its tip towards the cylindrical base 32. In an embodiment, the end of the pointy end 33 that is connected to the base 32 has the same diameter as the base 32 so as not to form any shoulder or flat surface at the junction between the base 32 and the pointy end 33. In another embodiment, the leading member 31 is constituted solely of a conical body. Consequently, the prominent end of the leading member 31, i.e., the pointy end 33, may rotate due to the circular cross-section.

Anti-rotation features may be present on the spike 30 so as to preclude rotation of the attachment member 25 relative to the bone once the spike 30 is fully inserted into the bone. The rotation may be about the central axis of the leading member 31 due to the circular cross-section of the leading member 31. In the embodiment, the anti-rotation features are fins 34. Three fins 34 are visible in FIG. 2 and are spaced 90 degrees apart on the cylindrical base 32, which may imply that a fourth fin may be present. However, it may suffice to have a single one of the fins 34, or other anti-rotation features. The anti-rotation feature may be defined as a lateral projection from a surface of circular cross-section. Other anti-rotation features may include an oval cross-section at a second penetration segment of the spike 30 (the first penetration segment defined by the pointy end 33). The fins 34 may have an angled edge 34A. In an embodiment, the angle is from 5 degrees to 30 degrees from the central axis X. The angled edge 34A may be followed up by a straight portion as shown (generally parallel to the central axis X, or at a flaring angle from 1 degree to 10 degrees), although this is not necessary. The angled edge 34a of the fins 34 may project radially from the base 32. Once the fins 34 penetrates the bone, the spike 30 is blocked from the rotating about its central axis. In an embodiment, the leading member 31 has a portion projecting beyond the anti-rotation feature(s) along the central axis X, as part of a first penetration segment of the spike 30, with a second penetration segment bound by the anti-rotation feature. The portion projects by a distance of at least 1 mm, and may be in a range of 1 mm to 15 mm, from the anti-rotation feature(s), although it may be longer. The leading member 31 have a maximum diameter ranging from 1 mm to 8 mm.

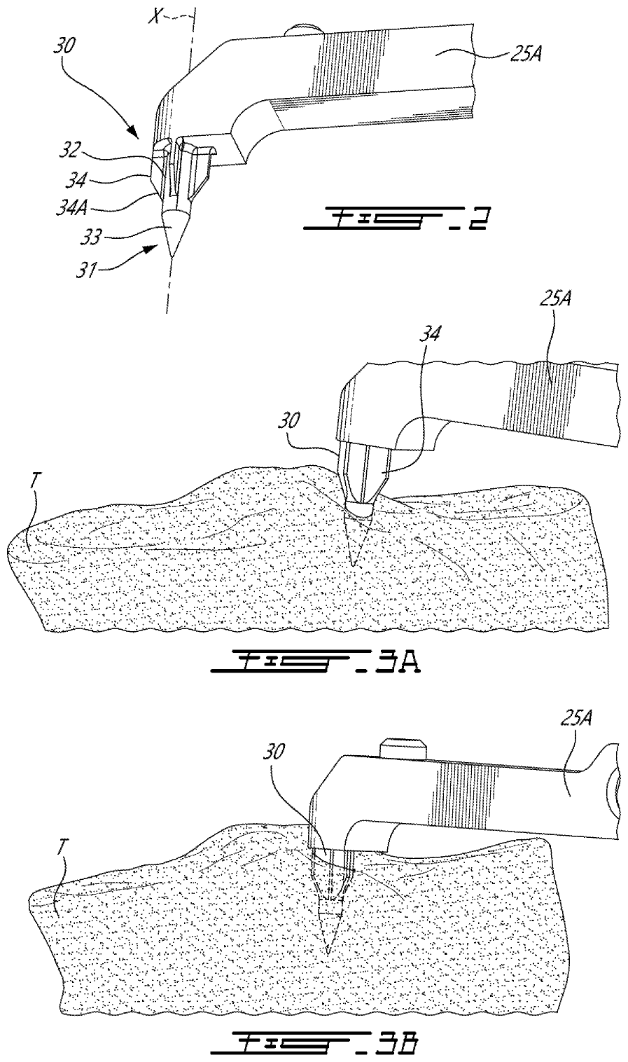

Now that the various components of the bone axis digitizer device 10 have been described, its installation onto a bone is set out. Referring to FIG. 1, the bone axis digitizer device 10 is installed on the bone in the manner shown in FIG. 1. This may include attaching the device 10 to the tibia T with the clamp 22 applied against the malleoli. In this step of attachment, the clamp 22 may be displaced and locked onto the main arm 21, the clamp 22 may be spread open and biased against the malleoli, etc. In the process, the position of the attachment member 25 relative to the main arm 21 may be adjusted so as to have the spike 30 contact the bone, such as the articular surface of the tibia T, for instance at a predetermined mechanical axis entry point. The relative positions of the clamp 22 and/or of the attachment member 25 on the main arm 21 may be manually adjusted, so as to visually align the main arm 21 with the tibia (or other bone in another application), such that the longitudinal axis of the bone is generally parallel to the main arm 21. The guidance provided by the inertial sensor unit 24 may also be used to guide the moving around of the spike 30 to be positioned at the mechanical axis entry point. In the embodiment, the user relies on a physical landmark to position the spike 30. With the attachment member 25 still in translational relation on the main arm 21, the spike 30 may be impacted into the tibia T. This may be achieved with any impacting tool, or with sufficient manual force. As a result, the pointy end 33 penetrates the bone, e.g., the tibial plateau. This is shown, for example, in FIG. 3A. In the embodiment, this is viewed as a first step in that the pointy end 33 has penetrated, as may have a portion of the cylindrical base 32. However, in this first step, the anti-rotation feature(s), e.g., fins 34, has not yet penetrated. As a result, it may still be possible to rotate the attachment member 25 relative to the tibia T, as only the round part of the spike 30 is in the bone. Hence, a manual adjustment of the orientation of the bone axis digitizer device 10 is possible, with the spike 30 rotating on itself. The penetration of the pointy end 33 only may enable subsequent adjustments of the position and/or orientation the guide frame 20, to achieve a desired orientation of the guide frame 20 on the bone, such as by achieve a parallel relation between the main arm 21 and bone (e.g., mechanical axis of the tibia T).

Once a desired orientation has been reached, the spike 30 may be further impacted into the tibia T. Through this second step of impacting, anti-rotation features, here the fins 34, penetrate the bone and/or cartilage. The angled edges 34a may facilitate the penetration of the fins 34. Once the fins 34 have penetrated the bone the bone (e.g., cortical bone) and/or cartilage, the spike 30 is blocked from rotating on itself relative to the bone. The spike 30 may be said to have a first penetration segment, having a first length, and a second penetration segment, having a second length, with the second penetration segment having an anti-rotation feature. The first penetration segment may be said to project axially beyond second penetration segment, in a penetration direction (e.g., central axis X). The first penetration segment may be round is cross-section, to which the penetration direction is normal, to allow rotation.

Therefore, the spike 30 described herein may be the sole attachment member at one end of the bone axis digitizer device 10, in contrast to devices having two distinct points of penetration. The spike 30 may be described as a position and orientation setting attachment component. At an end, the bone axis digitizer device 10 has only one bone attachment component, only one bone penetrating component, may reduce the number of parts, and may stiffen the point of connection of the bone axis digitizer device 10 at one end of the bone.

The spike 30 may be generally described as being for the bone axis digitizer device 10, having a central member having a pointy end configured for penetrating a bone or cartilage, the central member defining a central axis; one or more anti-rotation features projecting laterally from a surface of the central member. The central member has a first penetration segment and a second penetration segment, the first penetration segment configured for leading a penetration, and the second penetration segment having the at least one anti-rotation feature. The spike 30 may be said to be the single or only penetration portion of the device 10 at one end of the device 10.

The spike 30 may be related to a method for installing a bone axis digitizer device 10 on a bone, which may be included penetrating a bone and/or cartilage with a pointy end of a spike such that a first penetration segment of the spike penetrates the bone and/or cartilage; adjusting an orientation of the bone axis digitizer device by rotation of the spike relative to the bone; and further penetrating the bone and/or cartilage with a second penetration segment of the spike, the second penetration segment having an anti-rotation feature blocking rotation of the spike relative to the bone.

### EXAMPLES

The following examples can each stand on their own, or can be combined in different permutations, combinations, with one or more of other examples.

Example 1 is a bone axis digitizer device comprising: a main arm configured to extend along a bone; a clamp at an end portion of the main arm, the clamp configured to clamp to an anatomical portion; an attachment member at another end portion of the main arm; and a spike projecting from the attachment member, the spike including a leading member having a pointy end configured for penetrating a bone or cartilage, the leading member defining a penetration axis, and at least one anti-rotation feature projecting laterally from a surface of the leading member, wherein the spike has a first penetration segment and a second penetration segment, the first penetration segment including the pointy end and configured for leading a penetration of the spike in the bone or cartilage, and the second penetration segment having the at least one anti-rotation feature; wherein the bone axis digitizer device is configured to receive an inertial sensor unit thereon.

In Example 2, the subject matter of Example 1 includes, wherein the attachment member is connected to the main arm by a translational joint.

In Example 3, the subject matter of Example 1 includes, wherein the clamp is connected to the main arm by a translational joint.

In Example 4, the subject matter of Example 1 includes, wherein the main arm has a support for releasable connection of the inertial sensor unit to the main arm.

In Example 5, the subject matter of Example 1 includes, wherein the main arm is parallel to the penetration axis.

In Example 6, the subject matter of Example 1 includes, wherein the leading member is centered in the spike.

In Example 7, the subject matter of Example 1 includes, wherein the pointy end is part of a conical portion.

In Example 8, the subject matter of Example 7 includes, wherein the leading member has a cylindrical portion at an end of the conical portion.

In Example 9, the subject matter of Example 8 includes, wherein the at least one anti-rotation feature projects laterally from the cylindrical portion.

In Example 10, the subject matter of Example 1 includes, wherein the at least one anti-rotation feature is a fin.

In Example 11, the subject matter of Example 10 includes, wherein the fin has an angled edge tapering to the leading member toward the pointy end.

In Example 12, the subject matter of Example 10 includes, wherein the angled edge has an angle ranging from 5 degrees to 30 degrees of the penetration axis.

In Example 13, the subject matter of Example 10 includes, wherein the spike has four of the fins, the fins being equidistantly spaced around the leading member.

In Example 14, the subject matter of Example 1 includes, wherein the spike has a monoblock construction.

In Example 15, the subject matter of Example 1 includes, wherein the first penetration segment has a length from 1 to 15 mm, inclusively, along the penetration axis.

In Example 16, the subject matter of Example 1 includes, wherein the leading member has a maximum diameter ranging from 1 to 8 mm, inclusively.

Example 17 is a method for installing a bone axis digitizer device on a bone comprising: penetrating a bone and/or cartilage with a pointy end of a spike such that a first penetration segment of the spike penetrates the bone and/or cartilage; adjusting an orientation of the bone axis digitizer device by rotation of the spike relative to the bone; and further penetrating the bone and/or cartilage with a second penetration segment of the spike, the second penetration segment having an anti-rotation feature blocking rotation of the spike relative to the bone.

In Example 18, the subject matter of Example 17 includes, adjusting a length of the bone axis digitizer device after penetration of the first penetration segment into the bone, and before penetration of the second penetration segment.

In Example 19, the subject matter of Example 18 includes, wherein adjusting a length and adjusting an orientation includes aligning the bone axis digitizer device parallel to a bone.

In Example 20, the subject matter of Example 17 includes, wherein penetrating the bone and/or cartilage with the pointy end include penetrating the bone and/or cartilage at an entry point of a mechanical axis.

In Example 21, the subject matter of Example 20 includes, wherein penetrating the bone and/or cartilage at the entry point of the mechanical axis includes penetrating the bone and/or cartilage in the tibia.

1. A spike for a bone axis digitizer device comprising:
a leading member having a pointy end configured for penetrating a bone or cartilage, the leading member defining a penetration axis; and
at least one anti-rotation feature projecting laterally from a surface of the leading member;
wherein the spike has a first penetration segment and a second penetration segment, the first penetration segment including the pointy end and configured for leading a penetration of the spike in the bone or cartilage, and the second penetration segment having the at least one anti-rotation feature.

2. The spike according to claim 1, wherein the leading member is centered in the spike.

3. The spike according to claim 1, wherein the pointy end is part of a conical portion.

4. The spike according to claim 3, wherein the leading member has a cylindrical portion at an end of the conical portion.

5. The spike according to claim 4, wherein the at least one anti-rotation feature projects laterally from the cylindrical portion.

6. The spike according to claim 1, wherein the at least one anti-rotation feature is a fin.

7. The spike according to claim 6, wherein the fin has an angled edge tapering to the leading member toward the pointy end.

8. The spike according to claim 6, wherein the angled edge has an angle ranging from 5 degrees to 30 degrees of the penetration axis.

9. The spike according to claim 6, wherein the spike has four of the fins, the fins being equidistantly spaced around the leading member.

10. The spike according to claim 1, wherein the spike has a monoblock construction.

11. The spike according to claim 1, wherein the first penetration segment has a length from 1 to 15 mm, inclusively, along the penetration axis.

12. The spike according to claim 1, wherein the leading member has a maximum diameter ranging from 1 to 8 mm, inclusively.

13. A bone axis digitizer device comprising:
a main arm configured to extend along a bone;
a clamp at an end portion of the main arm, the clamp configured to clamp to an anatomical portion;
an attachment member at another end portion of the main arm; and
a spike projecting from the attachment member, the spike including a leading member having a pointy end configured for penetrating a bone or cartilage, the leading member defining a penetration axis, and at least one anti-rotation feature projecting laterally from a surface of the leading member, wherein the spike has a first penetration segment and a second penetration segment, the first penetration segment including the pointy end and configured for leading a penetration of the spike in the bone or cartilage, and the second penetration segment having the at least one anti-rotation feature;
wherein the bone axis digitizer device is configured to receive an inertial sensor unit thereon.

14. The bone axis digitizer device according to claim 13, wherein the attachment member is connected to the main arm by a translational joint.

15. The bone axis digitizer device according to claim 13, wherein the clamp is connected to the main arm by a translational joint.

16. The bone axis digitizer device according to claim 13, wherein the main arm has a support for releasable connection of the inertial sensor unit to the main arm.

17. The bone axis digitizer device according to claim 13, wherein the main arm is parallel to the penetration axis.

18. The bone axis digitizer device according to claim 13, wherein the leading member is centered in the spike.

19. The bone axis digitizer device according to claim 13, wherein the pointy end is part of a conical portion.

20. The bone axis digitizer device according to claim 19, wherein the leading member has a cylindrical portion at an end of the conical portion.
